(19) United States
(12) Patent Application Publication
(10) Pub. No.: US 2007/0053975 A1
(43) Pub. Date: Mar. 8, 2007

(54) Ramipril formulation

(75) Inventors: Paul Harrison, Dublin (IE); Anna Marie Power, Dublin (IE)

Correspondence Address: STERNE, KESSLER, GOLDSTEIN & FOX PLLC, 1100 NEW YORK AVENUE, N.W., WASHINGTON, DC, 20005, US

(73) Assignee: Selamine Limited, Dublin (IE)

(21) Appl. No.: 11/509,032

(22) Filed: Aug. 24, 2006

(30) Foreign Application Priority Data
Sep. 6, 2005 (GB) 05181292.2

(51) Int. Cl. A61K 9/20; A61K 31/403; A61K 38/05

(52) U.S. Cl. 424/464; 514/18; 514/412

(57) ABSTRACT

A Ramipril formulation rapidly disintegrates after ingestion and exhibits substantially no food effect.

### FIELD OF INVENTION

The present invention relates to a dosage form of Ramipril and also to methods of use. In particular, although not exclusively, the present invention relates to formulations for treating or preventing various disease states involving the administration of Ramipril, especially when a patient is in the fed state.

### BACKGROUND OF THE INVENTION

Ramipril, the United States Adopted Name (USAN) for (2S,3aS,6aS)-1[(S)-N-[(S)-1-carboxy-3-phenylpropyl]alanyl]octahydrocyclopenta[b]pyrrole-2-carboxylic acid, 1-ethyl ester (CAS Number 087333-19-5) is an angiotensin converting enzyme (ACE) inhibitor having the chemical structure shown below (I).

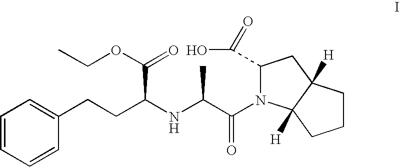

Ramipril and its acid are taught in EP 0 097 022. Ramipril has been used for the treatment of hypertension, heart failure, stroke, myocardial infarction, diabetes and cardiovascular disease. Ramipril may also reduce the risk of further strokes, heart attacks and cognitive impairment among stroke patients.

The absorption and bioavailability of a therapeutic agent may be affected by the presence of food in the gastrointestinal tract. Often, the gastric residence time of an orally administered drug is longer in the presence of food than in the absence. If the bioavailability of a drug is significantly affected by the presence of food in the gastrointestinal tract the drug may be said to exhibit a ‘food effect’.

Food effects usually mean that there is risk associated with administering a drug to a patient who has eaten recently. The particular type of dosage form used, the pH of the stomach and the susceptibility of actives to metabolism by liver enzymes all affect the bioavailability of actives. Sometimes, absorption of actives into the bloodstream may be limited to such an extent that a patient receives a sub-optimal dosage.

There is no way to predict with certainty whether a particular active will exhibit a food effect. For example, in the presence of food the absorption of aspirin is delayed, ampicillin is unaffected and diazepam is increased. In the presence of bergamottin, a compound seen in grapefruit, some compounds are even more active than normal.

Peak plasma concentrations are generally reached within one hour of oral administration of Ramipril if the patient is in the fasted state. In the case of Ramipril capsules (Altace®), the ‘patient leaflet information’ informs the patient that the absorption is affected by the presence of food in the gastrointestinal tract. In order to obtain the maximum clinical benefit patients should only be medicated when in the fasted state, i.e. at least one hour before or two hours following a meal.

It would be useful if Ramipril could be administered with no practical ‘food effect’ to patients that have eaten recently.

It is an object of the invention to provide a dosage form for Ramipril which can be administered to patients whether or not they have eaten whilst still maintaining a desired absorption profile.

### SUMMARY OF THE INVENTION

In a first aspect the invention provides Ramipril formulations that display rapid disintegration upon administration.

The term ‘rapid disintegration’ applies especially to those compositions that completely disintegrate in less than 15 minutes in purified water in accordance with the USP method over the range of viscosities anticipated in the stomach (i.e. from water to 5% Methocel E5 in water)

The tablet is preferably a “dispersible tablet” according to the European Pharmacopoeia, i.e. it disintegrates within 3 minutes when examined by the test for disintegration of tablets and capsules (2.9.1) using water at 15-25° C. Specific tablets of the invention have been found to disintegrate within 1½ minutes.

The formulations of the invention contain disintegrants of types and in quantities that achieve the disintegration profile specified. One of skill in the art will be familiar with amounts and types of disintegrants to use without resorting to undue experimentation. Suitable disintegrants include croscarmellose cellulose, crospovidone, sodium starch glycollate, low substituted hydroxypropylcellulose, and starches.

In another aspect the invention relates to a formulation comprising Ramipril which gives dissolution within minutes of administration as measured using the model systems described herein.

Preferably the dissolution level at 10 minutes after administration is 92%, more preferably 94% and most preferably 96% or greater.

Formulations of the invention also preferably give 98% dissolution within 20 minutes of administration and/or 99% within 20 minutes of administration. The most preferred formulations of the invention give substantially 100% dissolution within 30 minutes of administration.

The model system used to measure the dissolution utilised 10 mg Ramipril formulations studied using USP method II, in 500 mL of 5% Methocel E5 in water with stirring at 50 rpm.

The invention also provides a Ramipril containing formulation giving dissolution in vivo which is sufficiently rapid that presence or absence of food in the gastrointestinal tract does not substantially alter absorption of the Ramipril.

Formulations of the invention have been found to disintegrate rapidly and meet this criteria. In tests, absorption of Ramipril was measured by administering oral doses to patients with 200 mL water. Blood samples were withdrawn prior to dosing, and at 0.5, 1, 2, 3, 4, 6, 9, 12, 24, and 48 hours post-dosing. Serum Ramipril concentration was determined using a high performance liquid chromatography (HPLC) assay.

Formulations of the invention are generally regarded as providing Ramipril absorption that is not substantially altered by presence or absence of food either when peak plasma concentration of Ramipril in fed patients is not less than a third, preferably not less than a half of the peak plasma concentration in fasted patients, or when median time to maximum plasma concentration is not increased by more than 4, preferably 3, more preferably 2 fold and most preferably not more than 50%. Fed patients have eaten within an hour before or up to two hours after receiving the Ramipril.

This invention hence provides an oral dosage form of Ramipril which can be administered to a mammal (including humans) that has eaten and which exhibits substantially no adverse food effect.

In a further aspect, this invention provides a specific oral Ramipril dosage form which does not exhibit an adverse food effect. The dosage form comprises Ramipril and a pharmaceutically acceptable carrier, as hereinafter further detailed and described.

The dosage form is in the form of a tablet including both swallowable-only and chewable forms.

In a further aspect, this invention provides a method for treating or preventing a disease in a mammal selected from the group consisting of hypertension, heart failure, stroke, myocardial infarction, diabetes and cardiovascular disease or for reducing the risk of further strokes, heart attacks and cognitive impairment among stroke patients comprising administering to a mammal in need of such treatment, a pharmaceutically effective amount of Ramipril in an oral dosage form according to the invention. Preferably the mammal has eaten, and reference to a mammal (including humans) that has “eaten” means that the mammal has eaten food of any sort within one hour prior to dosing or up to two hours after dosing.

This invention provides an oral dosage form of Ramipril which can be administered to a mammal (including humans) that has eaten and which exhibits substantially no adverse food effect. The dosage form exhibits a substantially unaltered extent of absorption defined as the area under the curve of a drug plasma concentration against time curve in the fed and fasted state, and a substantially unaltered rate of drug absorption defined by time to maximum drug plasma concentration and peak concentration between the fasted and fed state.

The rapidly disintegrating oral dosage form of Ramipril comprises Ramipril and pharmaceutically acceptable carriers, as herein further detailed and described as part of the invention. The dosage form is in the form of a tablet (including both swallowable and chewable forms).

In dosage forms according to the invention, absence of a substantial food effect is surprising as alternative conventional formulations in the commercial domain have been reported to have altered pharmacokinetic attributes in the fed and fasted state e.g. see Altace® Patient Information Leaflet.

In a further aspect, this invention provides a therapeutic package suitable for commercial sale, comprising a container, an oral dosage form of Ramipril which does not exhibit an adverse food effect contained therein, and, associated with said container, written matter non-limited as to whether the dosage form can be taken with or without food.

For purposes of this invention Ramipril may be administered alone or in combination with other therapeutic agents. In one embodiment Ramipril is co-administered with a diuretic agent, preferably the diuretic is selected from hydrochlorothiazide or piretanide.

That a dosage form according to the invention does not exhibit an adverse food effect is further surprising in view of the fact that Ramipril is unstable at low (acid) pH, on the order of the acidity encountered at the pH of stomach acid. The inventors have demonstrated that Ramipril breaks down if exposed to stomach juices which inherently exhibit acid pH. Thus, without being bound to any mechanism of action, it is surprising that rapid disintegration in the GI tract appears to be of importance to the invention.

Ramipril is typically present in formulations according to the invention in an amount of from about 1.25 mg to about 10 mg; other formulations may have 2.5 mg or 5 mg per tablet. The amount of active can be adjusted to be outside these limits depending, for example, on the size of the animal subject being treated (e.g., a horse). The term ‘Ramipril’ includes all the pharmaceutically acceptable versions thereof, e.g. salts, esters, clathrates thereof, and also anhydrous as well as hydrated forms.

A conventional dosage form can be construed to be a formulation where no novel adjuvant/excipient or particular in vitro specification has been claimed to benefit the pharmacokinetic profile of the drug substance after administration. An in vitro specification is more commonly defined as the time in which the drug dissolves, under controlled agitation in a physiologically related aqueous solution. The most common in vitro test is known as the dissolution test and is fully described in USP.

The pharmacokinetic attributes that describe the ‘drug availability’ in the fed and fasted state can be quantified by measuring the plasma concentration of the drug substance against time in a population of subjects. The total amount available in the plasma, available for the therapeutic effect, is quantified by the area under the curve (AUC) of the plasma time plot. The rate of availability of the therapeutic dose of the drug in the plasma, and consequent therapeutic activity will be related to the time of and value of the peak plasma concentration.

A pharmacokinetic study was conducted that assessed the food effect with two formulations. The mean key pharmacokinetic parameters of fed and fasted studies for Ramipril (Formulation B is a prior art formulation; Formulation A is in accordance with the invention) are presented in tables 1 and 2.

TABLE 1
Mean Pharmacokinetic Results- Fasted Condition

| Pharmacokinetic Parameter | Formulation A | | Formulation B | |
|---|---|---|---|---|
| AUC | 16.77 | ng · hr/ml | 16.40 | ng · hr/ml |
| Peak Concentration | 27.58 | ng/ml | 25.11 | ng/ml |
| Median time to max | 0.50 | hr. | 0.50 | hr |
| concentration | | | | |

TABLE 2
Mean Pharmacokinetic Results- Fed Condition

| Pharmacokinetic Parameter | Formulation A | | Formulation B | |
|---|---|---|---|---|
| AUC | 19.23 | ng · hr/ml | 16.15 | ng · hr/ml |
| Peak Concentration | 15.1 | ng/ml | 6.58 | ng/ml |
| Median time to max | 0.67 | hr | 2.25 | hr |
| concentration | | | | |

It can be inferred from the two studies that formulation does not substantially alter drug absorption (as defined by pharmacokinetic parameters) in the fasted state but is substantially different in the fed state. Both the extent and rate of absorption of Formulation B is reduced in the fed state compared to Formulation A (of the invention).

The definition of “fed” in relation to this study is a US Food and Drug Administration (FDA)-recommended standard high fat breakfast ingested 30 minutes before administration of the oral dosage form in 240 ml of water.

Table 1 & 2 indicate that availability of the drug is affected by formulation. Rapid disintegration of formulation A improves the availability of the drug for absorption in the fed state.

An in vitro assessment of the dissolution profile of formulation A and B was performed in physiological related media in accordance with USP. The results are presented below.

TABLE 3
Ramipril 10 mg USP method II 50 rpm volume 500 mL Medium
0.1 NHCl

| % dissolved | 10 mins | 20 mins | 30 mins |
|---|---|---|---|
| Formulation A | 96 | 100 | 100 |
| Formulation B | 87 | 97 | 97 |

The dissolution from the dosage form will be dependent on the surface area available according to the Noyes-Whitney equation. By increasing the surface area available for dissolution, by including additives in a formulation to aid disintegration of the oral dosage form, it is possible to obtain rapid dissolution. Ingredients known as disintegrants are therefore included in oral dosage forms of the invention to ensure rapid dissolution.

Typical disintegrant include starch, and derivatives thereof, and cross linked polymers such as cross linked povidone and sodium carboxymethylcellulose, starches, low substituted hydroxypropylcellulose (L-HPC), carbonate salts, aluminium magnesium silicate and silicon dioxide

Disintegrants work by two interrelated mechanisms, by wicking water into the tablet core increasing the surface area available to the aqueous environment and by swelling on uptake of water.

In order to further demonstrate the properties of the solid dosage form of the present invention, a number of additional tests were performed comparing the disintegration and dissolution of the solid dosage form with known solid dosage forms.

Three additional tests were performed:
(i) A model for high agitation in a “fasted state”, where the tablet must disintegrate at physiological temperature (37° C.) in water within 3 minutes in a standard USP disintegration test;
(ii) A model for high agitation in a “fed state”, where the tablet must disintegrate at physiological temperature (37° C.) in a 5% w/v Methocel E5 solution in water within 15 minutes in a standard USP disintegration test; and
(iii) A model for low agitation in a “fed state”, where the tablet must disintegrate at physiological temperature (37° C.) in a 5% w/v Methocel E5 solution in water within 30 minutes in a modified USP dissolution test.

The modified USP dissolution test in (iii) was as follows:

A screen circle of equivalent mesh size to USP disintegration basket was manufactured to such a diameter that it sat equidistant from the bottom of the paddle to the base of the dissolution pot. The distance was 12.5 cm from the bottom of the pot and 12.5 cm from the paddle. A “capsule sinker” was placed securely fastened to the circular mesh. The purpose of the sinker was to keep the tablet/capsule in a fixed position for the test.

The tablets/capsules to be tested were placed in the sinker in such a manner that the tablet/capsule was at right angles to the arms of the sinker, and positioned so that the tablet/capsule was midway from the centre to the outer point of the circular screen.

The USP dissolution pots were filled with 500 ml of 5% Methocel ES solution and heated to 37° C. Tablets/capsules secured in the sinker on the mesh were then dropped into the filled dissolution pot and the tablets/capsules adjusted so that the mesh was positioned horizontally. The paddles were immediately lowered and stirring commenced at 50 rpm.

The time taken for the tablet/capsule to disintegrate was recorded and was determined to be the time taken for the entire tablet to pass through the mesh.

The results were as follows:

TABLE 4
Model for high agitation in a “fasted state”
Test: USP Disintegration method (without discs) Medium: Water
Pass Criteria: less than 3 minutes

| | Batch | | | | Pass/ |
|---|---|---|---|---|---|
| Description | Number | Formulation | Water | | Fail |
| Ramipril 10 mg T | — | SDF of invention | 1 | min | Pass |
| Ramipril 10 mg T | — | SDF of invention | 1 | min | Pass |
| Ramipril 10 mg T | — | SDF of invention | 1 | min | Pass |
| Ramipril 2.5 mg T | — | SDF of invention | 45 | sec | Pass |
| Ramipril 2.5 mg T | — | SDF of invention | 50 | sec | Pass |
| Ramipril 2.5 mg T | — | SDF of invention | 45 | sec | Pass |
| Ramipril 1.25 mg T | — | SDF of invention | 40 | sec | Pass |
| Ramipril 1.25 mg T | — | SDF of invention | 40 | sec | Pass |
| Ramipril 1.25 mg T | — | SDF of invention | 40 | sec | Pass |
| Ramipril 5 mg T | — | SDF of invention | 50 | sec | Pass |
| Ramipril 5 mg T | — | SDF of invention | 45 | sec | Pass |
| Ramipril 5 mg T | — | SDF of invention | 50 | sec | Pass |
| Ramipril 10 mg C | B4007 | Generic version | 7 | min | Fail |
| Altace ® 5 mg C | TBE | Brand US | 6 | min | Fail |
| Altace ® 2.5 mg C | 1094258 | Brand US | 6 | min | Fail |
| Altace ® 10 mg C | 2775 | Brand US | 7 | min | Fail |
| Altace ® 10 mg C | 40L23J | Brand Portugal | 6 | min | Fail |
| Tritace ® 5 mg T | 502708 | Brand Brazil | 3 | min | Pass |
| Tritace ® 5 mg T | 58051 | Brand Australia | 1 | min | Pass |
| Tritace ® 2.5 mg T | 57178 | Brand Australia | 1 | min | Pass |
| Tritace ® 10 mg T | D457 | Brand Sweden | 1 | min | Pass |

T = tablet
C = capsule
SDF = solid dosage form

TABLE 5
model for high agitation in a “fed state”
Test: USP Disintegration method (without discs) Medium: 5% Methocel E5
Pass Criteria: less than 15 minutes

| Description | Batch Number | Formulation | 5% Methocel E5 | | Pass/Fail |
|---|---|---|---|---|---|
| Ramipril 10 mg T | — | SDF of invention | 13 | min | Pass |
| Ramipril 10 mg T | — | SDF of invention | 13 | min | Pass |
| Ramipril 10 mg T | — | SDF of invention | 14 | min | Pass |
| Ramipril 2.5 mg T | — | SDF of invention | 10 | min | Pass |
| Ramipril 2.5 mg T | — | SDF of invention | 10 | min | Pass |
| Ramipril 2.5 mg T | — | SDF of invention | 10 | min | Pass |
| Ramipril 1.25 mg T | — | SDF of invention | 8 | min | Pass |
| Ramipril 1.25 mg T | — | SDF of invention | 8 | min | Pass |
| Ramipril 1.25 mg T | — | SDF of invention | 8 | min | Pass |
| Ramipril 5 mg T | — | SDF of invention | 12 | min | Pass |
| Ramipril 5 mg T | — | SDF of invention | 12 | min | Pass |
| Ramipril 5 mg T | — | SDF of invention | 12 | min | Pass |
| Ramipril 10 mg C | B4007 | Generic version | 22 | min | Fail |
| Altace ® 5 mg C | TBE | Brand US | 11 | min | Pass |
| Altace ® 2.5 mg C | 1094258 | Brand US | 11 | min | Pass |
| Altace ® 10 mg C | 2775 | Brand US | 18 | min | Fail |
| Altace ® 10 mg C | 40L23J | Brand Portugal | 16 | min | Fail |
| Tritace ® 5 mg T | 502708 | Brand Brazil | 32 | min | Fail |
| Tritace ® 5 mg T | 58051 | Brand Australia | NLT 45 | min | Fail |
| Tritace ® 2.5 mg T | 57178 | Brand Australia | NLT 45 | min | Fail |
| Tritace ® 10 mg T | D457 | Brand Sweden | NLT 45 | min | Fail |

T = tablet
C = capsule
NLT = not less than
SDF = solid dosage form

TABLE 6
model for low agitation in a “fed state”
Test: USP Dissolution Studies - Modified Medium: 5% Methocel E5 50 rpm
Pass criteria: Not more than 30 minutes:

| Description | Batch Number | Formulation | 5% Methocel E5 | Pass/Fail |
|---|---|---|---|---|
| Ramipril 1.25 mg T | — | SDF of invention | 14 min | Pass |
| Ramipril 5 mg T | — | SDF of invention | 16 min | Pass |
| Ramipril 2.5 mg T | — | SDF of invention | 10 min | Pass |
| Ramipril 10 mg T | — | SDF of invention | 18 min | Pass |
| Ramipril 10 mg C | M5022 | Generic version | NLT 2 hours | Fail |
| Altace ® 5 mg C | TBE | Brand US | NLT 2 hours | Fail |
| Altace ® 2.5 mg C | 1094258 | Brand US | NLT 2 hours | Fail |
| Altace ® 10 mg C | 2775 | Brand US | NLT 2 hours | Fail |
| Altace ® 10 mg C | 40L23J | Brand Portugal | 57 minutes | Fail |
| Tritace ® 5 mg T | 502708 | Brand Brazil | NLT 2 hours | Fail |
| Tritace ® 5 mg T | 58051 | Brand Australia | NLT 2 hours | Fail |
| Tritace ® 2.5 mg T | 57178 | Brand Australia | 1 hour 15 min | Fail |
| Tritace ® 10 mg T | D457 | Brand Sweden | NLT 2 hours | Fail |

T = tablet
C = capsule
NLT = not less than
SDF = solid dosage form

The results show that in a state of low agitation formulations of the present invention disintegrate more rapidly than the commercial formulations tested. Because of the retarded disintegration under low agitation, it would be expected that the rate of absorption of the drug from the commercial formulation would be slower in the state of low agitation that is likely to occur after a meal. The in vivo study supports this finding as the T max for the commercial product was considerably longer than for the formulation of the present invention (2.25 hr versus 0.67 hr—see table 2).

It is interesting to note that the disintegration of the formulations of the present invention is not markedly altered in the viscous medium in the low agitation and high agitation models. This would indicate that the product has been well formulated. The motility of the stomach in a viscous state is unlikely to substantially affect the rate of absorption of the drug.

The disintegration results for the Tritace® tablets are of particular note. The tablets disintegrate rapidly in the aqueous media, and very slowly in the viscose media. The disintegration process and probably the absorption process of Ramipril from this commercial product will be highly sensitive to stomach content viscosity.

A bioavailability study in the fasted state was performed on the 5 mg Tritace® brand in Brazil against a formulation of the present invention.

The following results were obtained:

TABLE 7
Fasted Patient Trial

| Pharmacokinetic | Formulation of the present | |
|---|---|---|
| Parameter | invention | Tritace ® product |
| AUC | 6.34 ng/hr · ml | 5.78 ng/ · hr ml |
| C max | 9.58 ng/ml | 8.47 ng/ml |
| T max | 0.49 hr | 0.67 hr |

The results indicate that even in the fasted state the formulation of the present invention demonstrated a faster rate of absorption, greater C max value and improved AUC.

If it is assumed that the rate limiting step to drug absorption of Ramipril is the disintegration of the dosage form and because the disintegration of Tritace® is very sensitive to viscosity, it can be surmised that the difference in the fasted state in this study could be assigned to a viscosity effect in the stomach. The viscosity of the stomach content in the fasted state is probably more akin to the viscous model than the aqueous model, or something in between.

It is clear from the data presented that the formulations of the present invention exhibit improved disintegration when compared with the other formulations, and this improvement rank correlates with the improved in vivo results. It also provides a logical explanation for the in vivo differences in products that rapidly dissolve and which are seemingly readily available for drug absorption as determined from the conventional USP disintegration and dissolution testing.

It is interesting to note from these studies that the disintegration of conventional formulations can be highly sensitive to formulation. This study is novel in that this is the first study where differences in the disintegration times of products have been correlated with in vivo data. The simplest correlation is the disintegration of the formulation in viscose media and the T max value of the product in vivo in the fed state.

In the fasted state the situation is more difficult to interpret. It is probable that the viscosity of the stomach content is not as high as in the in vitro test system, but not the same as a simple aqueous system. This would explain why the Altace® capsule product and formulations of the present invention exhibited similar in vivo T max values, whereas the Tritace® tablet and the formulations of the present invention differ. The reason for this is that the Tritace® tablet is extremely sensitive to viscosity and any increases even in the fasted state would potentially retard disintegration, and subsequent drug absorption.

It can be concluded that formulations of the present invention exhibit rapid disintegration in the in vitro models in aqueous and viscose media with differing degrees of agitation. This is not a feature of known formulations and affords the formulations a rapid rate of absorption that is not markedly affected by food.

According to a further aspect of the present invention, there is, therefore, provided a Ramipril formulation which disintegrates in less than 3 minutes in a model for high agitation in a fasted state, in less than 15 minutes in a model for high agitation in a fed state, and in less than 30 minutes in a model for low agitation in a fed state.

### EXAMPLES

The following examples are provided to illustrate the invention only and should not be construed as limiting the scope of the invention as claimed herein.

### Example 1

Formulation for 1.25 mg tablet containing Ramipril

| | |
|---|---|
| Ramipril | 0.56% |
| Calcium phosphate | 83.18% |
| Pregelatanised starch | 9.98% |
| Na croscarmellose | 2.99% |
| Mg stearate | 2.99% |
| Na lauryl sulphate | 0.30% |

### Example 2

Formulation for 2.5 mg tablet containing Ramipril

| | |
|---|---|
| Ramipril | 1.11% |
| Calcium phosphate | 82.71% |
| Pregelatanised starch | 9.93% |
| Na croscarmellose | 2.98% |
| Mg stearate | 2.98% |
| Na lauryl sulphate | 0.30% |

### Example 3

Formulation for 5 mg tablet containing Ramipril

| | |
|---|---|
| Ramipril | 2.22% |
| Calcium phosphate | 81.78% |
| Pregelatanised starch | 9.81% |
| Na croscarmellose | 2.94% |
| Mg stearate | 2.94% |
| Na lauryl sulphate | 0.29% |

### Example 4

Formulation for 10 mg tablet containing Ramipril

| | |
|---|---|
| Ramipril | 4.45% |
| Calcium phosphate | 79.92% |
| Pregelatanised starch | 9.59% |
| Na croscarmellose | 2.88% |
| Mg stearate | 2.88% |
| Na lauryl sulphate | 0.29% |

The invention thus provides rapidly disintegrating Ramipril-containing formulations which substantially avoid any food effect associated with fed-status of the patient.

1. A Ramipril formulation which disintegrates in less than 15 minutes in the USP disintegration test carried out over the anticipated viscosity range in the stomach and which disintegrates in less than 3 minutes in water in the USP disintegration test.

2. The formulation of claim 1, wherein at least 90% of the Ramipril is dissolved within 10 minutes of administration measured using the USP method, using 0.1NHCL, 50 rpm paddles in 500 ml.

3. A Ramipril containing formulation giving dissolution of Ramipril in vivo which is sufficiently rapid that presence or absence of food in the gastrointestinal tract does not substantially alter absorption of the Ramipril.

4. The Ramipril containing formulation of claim 3, wherein peak plasma concentration of Ramipril in fed patients is not less than a third that in fasted patients.

5. The Ramipril containing formulation of claim 3, wherein peak plasma concentration of Ramipril in fed patients is not less than a half that in fasted patients.

6. The Ramipril containing formulation of claim 3, wherein median time to maximum plasma concentration in fed patients is not increased by more than 4 fold in fasted patients.

7. The Ramipril containing formulation of claim 3, wherein median time to maximum plasma concentration in fed patients is not increased by more than 2 fold in fasted patients.

8. An oral Ramipril formulation which does not exhibit a food effect.

9. The formulation of claim 1 comprising a disintegrant.

10. The formulation of claim 9 wherein the disintegrant is selected from croscarmellose cellulose, crospovidone and sodium starch glycollate.

11. A Ramipril formulation which disintegrates in less than 3 minutes in a model for high agitation in a fasted state, in less than 15 minutes in a model for high agitation in a fed state, and in less than 30 minutes in a model for low agitation in a fed state.

12. A method for treating or preventing a disease in a mammal selected from the group consisting of hypertension, heart failure, stroke, myocardial infarction, diabetes and cardiovascular disease or for reducing the risk of further strokes, heart attacks and cognitive impairment among stroke patients comprising administering to a mammal in need of such treatment, the formulation of claim 1.

13. The method of claim 12, wherein the mammal has eaten.

14. A kit, comprising
a container,
an oral dosage form of Ramipril that does not exhibit a food effect, and
written matter non-limited as to whether the dosage form can be taken with or without food.

15. The kit of claim 14, wherein the oral dosage form is a formulation according to claim 1.
